(19) United States
(12) Patent Application Publication
(10) Pub. No.: US 2024/0139698 A1
(43) Pub. Date: May 2, 2024

(54) REACTOR SYSTEM FOR MIXING OPERATION AT PARTIAL LOAD

(71) Applicant: Casale SA, Lugano (CH)

(72) Inventor: Maurizio RIZZI, Villa Guardia (IT)

(21) Appl. No.: 18/278,453

(22) PCT Filed: Feb. 24, 2022
(86) PCT No.: PCT/EP2022/054698

§ 371 Date: Aug. 23, 2023

(30) Foreign Application Priority Data
Mar. 16, 2021 (EP) 21162870.6

(51) Int. Cl. B01J 8/04; B01J 8/00; C01C 1/04; C07C 29/152

(52) U.S. Cl. CPC B01J 8/0492; B01J 8/001; B01J 8/0496; C01C 1/0417; C07C 29/152; B01J 2208/00168; B01J 2208/00371; B01J 2208/00548; B01J 2208/00823; B01J 2208/00938

(57) ABSTRACT

A reactor system comprising a multibed catalytic converter including a mixing region upstream of a catalytic bed, the mixing region is arranged to mix a feed gas of the catalytic bed with a mixing gas, the mixing gas is introduced in the mixing region via a plurality of mixing gas feed lines, each of said lines includes at least one flow regulators device so that the amount of mixing gas admitted into the mixing region by each of the mixing gas feed lines is independently controlled.

### FIELD OF APPLICATION

The present invention concerns the field of reactor systems for industrial applications such as synthesis of ammonia and methanol. In particular, the present invention concerns a reactor system for improved mixing operation at partial load.

### PRIOR ART

Industrial synthesis reactors are generally conceived to run at full capacity or close to maximum capacity because the plant's economic profitability is expected to drop at reduced capacity.

In more details, at partial load the production rate of chemical converters is reduced and the yield of the chemical reaction may be lower than the expected because mass and heat transfer limitations may become important for example as a consequence of a not adequate mixing between the reactants in the catalytic beds.

A good mixing established inside the chemical converters is essential to guarantee a uniform temperature and composition profile. In the absence of good mixing, no or little reaction will take place. Furthermore, a uniform temperature and composition profile guarantees that the converters work as close as possible to the optimized profile exploiting at best the catalyst volumes.

In addition, a uniform temperature and composition profile prevents the formation of potential by-products, which can negatively affect the process yield and might create purification issues in downstream process units. Lastly, since most of the converters are operated via automatic control loops, it is essential to have reliable leading temperature signals to avoid loss of efficiency in the converter or even unwanted shutdown. Unfortunately, at reduced capacity or low turn down, due to fluid-dynamic reasons, the mixing devices arranged in the converter lose a lot of their effectiveness.

Typically, a suitable mixing between two or more gas reactants in a catalytic converter is achieved by imposing an adequate flow velocity of the reagents fed to the converter. When the operating condition of the converter switches from full to partial load, the flow velocity of reagents provided to the converter may drop under a threshold value where no satisfactory mixing condition can be established so that mass and heat transport limitations may arise thus limiting the maximum conversion achievable, the catalyst selectiveness or even the converter reliability.

Unfortunately, it is not always possible to operate the converters at full capacity. Typically, reactors that may be forced to operate under partial load conditions are the ammonia and the methanol converters when one of the reagents (e.g. hydrogen) is produced using apparatus powered by renewal energy sources. For example, hydrogen can be produced in a water electrolyzer powered by solar or wind energy. Such plants that exploit renewal energy sources to synthesize one of the reagents are known as green plants.

The green plants are of great interest because of the low operational cost and low pollution; for example, they do not produce CO₂ contrary to the conventional coal-based or natural gas-based plants. However, renewable sources like solar or wind are intrinsically subject to fluctuations, e.g. solar energy is not available during night time. Therefore, load fluctuations occurring during the production cycle must be expected and dealt with.

Unsatisfactory mixing conditions may be established not only in the catalytic beds of the converter but as well in the interbed cooling section of the reactor usually interposed between two subsequent catalytic beds. Typically, in the converter's interbed cooling section, the effluent of the catalytic bed is cooled before being fed to a further conversion step in a second bed. Usually, the cooling medium is a “fresh” reagent having a flow velocity that is high enough to promote a complete mixing with the effluent exiting the catalytic bed in a short amount of time.

Therefore, in light of the considerations above mentioned, it is highly desirable to find a reactor system for industrial applications where adequate mixing conditions can be established inside the reactor independently on the load applied or in other worlds independently on the flow circulating in the system. Additionally, it is highly desirable to find a method for controlling the flow rate of a gas circulating in a reactor system while granting satisfactory mixing conditions inside the reactor even at partial load.

WO 2013/097958 discloses a multi-bed catalytic converter with inter-bed cooling and a related process of conversion of reagents into products. U.S. Pat. No. 5,648,051 discloses an apparatus and method for quenching in hydroprocessing of a hydrocarbon feed stream. WO 99741329 discloses a hydroprocessing reactor and process with liquid quench. U.S. Pat. No. 7,275,565 discloses a multi-conduit multi-nozzle fluid distributor.

### SUMMARY OF THE INVENTION

The invention aims to overcome the above drawbacks of the prior art. In particular, the present invention aims to provide a novel reactor system configured to establish satisfactory mixing conditions inside a catalytic converter.

The above aim is reached with a reactor system according to claim 1.

The reactor system comprises a catalytic converter and a reagent feed line connected to said converter. The catalytic converter comprises one or more catalytic beds and a mixing region arranged upstream of a catalytic bed to mix the inlet feed of said catalytic bed with a mixing gas. The reactor system further comprises a mixing gas feed lines arranged to feed said mixing gas to the mixing region of the converter.

Each of said mixing gas feed lines includes at least one flow regulator device so that the amount of mixing gas admitted into the mixing region by each of the feed lines can be independently controlled.

Advantageously, the flow regulator devices allow to independently adjust the flow rate of the mixing gas circulating in each line so that the flow velocity of the mixing gas entering the mixing section is maintained in a target flow velocity range. The target velocity range aims to establish a complete and timely mixing between the inlet feed of said catalytic bed with the mixing gas so that a uniform temperature and composition profile is established in the resulting mixture before reaching the catalyst retained in the catalytic bed of the converter.

Additionally, by maintaining the flow velocity of the mixing gas entering the mixing section in a target range, a uniform distribution of the reactants over the catalyst surface can be established to minimize mass and heat transport limitation.

A further aim of the invention is to provide a method for adjusting the flow rate of a mixing gas circulating in a reactor system wherein the flow rate of the mixing gas fed into the mixing regions of the converter is adjusted by means of the flow regulator devices as a function of the load of the converter.

Advantageously, when the reactor system is subjected to partial load event so when the flow rate of the circulating gas fed to the reactor system decreases (even abruptly), the flow velocity of the makeup gas entering the converter can be maintained in the target velocity range just by acting on the flow regulator devices.

### PREFERRED EMBODIMENTS

According to a preferred embodiment of the invention, the catalytic converter is a multibed converter including a plurality of catalytic beds and a plurality of mixing regions wherein the number of mixing regions is equal to the number of catalytic beds.

Preferably each mixing region of the catalytic converter is arranged upstream a respective catalytic bed and each mixing region is connected to a respective mixing gas feed line.

According to an embodiment, the mixing region may be confined in a portion of the catalytic bed and arranged upstream of the catalyst so that the mixing between the inlet feed of the catalytic bed and the mixing gas occurs prior to reach the catalyst.

The reactor system comprises mixing gas feed lines configured to feed a mixing gas to the mixing region of the converter. Preferably, said mixing gas feed lines are branched off from a main header so that the mixing gas traversing the main header is split across the mixing gas feed lines. The amount of flow circulating in each mixing line may be controlled by a respective flow regulator device.

According to a preferred embodiment, each mixing region includes a mixing device connected to a respective mixing gas feed line.

Said mixing device is configured to distribute the mixing gas in the mixing region so as to establish satisfactory mixing conditions between the inlet feed of the catalytic bed and the mixing gas. Satisfactory mixing conditions are achieved by maintaining the flow velocity of the mixing gas entering the mixing region to a target value or to a target flow velocity range. The optimal flow velocity range is selected during operation taking into consideration the operating parameter of the converter and the properties of the mixing gas.

According to a preferred embodiment, the mixing device comprises a plurality of flow distributors; each flow distributor is in fluid communication with a respective mixing gas line so that the mixing gas enters the mixing region from separated mixing lines and each distributor is fed individually. The flow rate and therefore the flow velocity of the mixing gas entering the mixing section via each distributor can be regulated or adjusted by the flow regulator devices.

Preferably, the flow distributors are rings or toroids concentrically arranged over each other so to reduce the volume occupied by the mixing device inside the mixing section. This arrangement maximises the room available to the catalytic bed, i.e. the amount of catalyst that can be used.

Preferably, the flow distributors are connected to the mixing device via coaxially arranged tubes to feed the mixing gas to the flow distributors as independent streams. The coaxially arranged tubes allow minimising the volume occupied by the mixing device and reduce the fluid dynamic disturbance that the section of the flow distributors causes to the discharge of the inlet gas into the mixing region.

Preferably the cross-section area and the length of the coaxially arranged tubes are designed to achieve a comparable pressure drop (flowing resistance) when traversed by an equal flow of gas.

Preferably the flow regulator devices are valves that allow a precise regulation of flow (e.g. high quality control valves), preferably having the same relationship between the valve capacity and the plug's displacement (i.e. valve characteristic).

Preferably each valve is provided with its own actuator capable of receiving and translating the signals received from a programmable control unit or a distributed control system into a plug's displacement to close or partially close said valves.

According to a particularly preferred embodiment of the present invention, the total number of flow regulator devices arranged onto the mixing lines is equal to the number of flow distributors of the mixing devices. At least one flow regulator is arranged on each mixing line.

The flow distributor devices may be provided with circular or substantially circular openings having the same cross-sectional area available to the discharge of the mixing gas into the mixing section.

According to a preferred embodiment of the present invention, the reactor system comprises a programmable interface controller or a distributed control system that determines the plug's displacement of the valves to adjust the flow rate (flow velocity) of the mixing gas entering the mixing sections during partial load events.

Preferably, when the mixing gas circulating in the main header decreases, the programmable controller or the distributed control system send a signal to the actuators of the flow control devices so as to close or partially close at least one of the valves located on the mixing gas feed lines. Due to the latter operation, the flow velocity of the mixing gas entering the mixing sections is re-established to the optimal value or in the optimal range.

According to another particularly preferred embodiment of the present invention, the catalytic converter is a multibed converter exploited for the synthesis of ammonia or methanol provided with multiple catalytic beds and multiple mixing sections wherein each mixing section acts as a cooling zone wherein a cold stream is exploited to cool down a reagent mixture or a partially converted reagent mixture.

Preferably, the number of mixing lines connected to each mixing zone is selected as a function of the catalytic converter's operating conditions and depending on the fluid dynamic conditions within the mixing zone. As a general rule, increasing the number of mixing lines connected to a mixing zone results in a more accurate control of the flow velocity resulting in no flow velocity deviation or just in a minimal offset of the flow velocity from the setpoint value.

Additionally, increasing the number of mixing lines communicating within the mixing zone increases the system's ability to accommodate for low reductions in the flow rate of the mixing gas circulating in the reactor system.

Preferably, the most accurate control of the flow velocity of the gas mixture entering the catalytic converter may be required in the mixing section arranged upstream the first catalytic bed, wherein even a slight variation of the operating parameters from the target values may drastically and negatively affect the reaction yield.

According to a particularly preferred embodiment of the present invention, the mixing gas fed to the mixing section may be used as a quenching medium to cool down the inlet gas entering a catalytic bed, being the temperature of said quenching medium lower than the temperature of said inlet gas. According to this embodiment, the flow distributors devices are quench rings.

Preferably, the catalytic converter may comprise a heat exchanger arranged after a bed or enclosed around it so as to maintain a pseudo isothermal temperature profile across the reactive section (i.e. the catalytic bed).

According to an embodiment of the invention, when the catalytic synthesis is carried out via exothermic reactions, the reagent feed line carrying the fresh reagent may be arranged to cross the catalytic converter so to remove the reaction heat by indirect heat transfer from the catalytic bed to the reagent gas so to provide a heated fresh reagent gas at the inlet of the converter.

According to a preferred embodiment of the present invention, the reagent feed line is branched off from the main header to form a network of pipes with the mixing gas feed lines.

Another aspect of the invention is to provide a method for controlling the flow rate of a mixing gas circulating in a reactor system subjected to partial load events. The method of the present invention can be extended to any reactor systems comprising a converter wherein the pressure drop across the mixing section is dominant over the pressure drop measured across the mixing gas feed lines.

The method of the present invention is particularly suited for a reactor system comprising a catalytic converter that operated at high pressure so that the localised pressure drop across each valve is negligible over the absolute pressure of the reactor.

Particularly preferably, an embodiment of the invention is:
a reactor system comprising:
a catalytic converter;
a reagent feed line connected to said converter, wherein said converter comprises one or more catalytic beds and further comprises a mixing region arranged upstream of a catalytic bed to mix the inlet feed of said catalytic bed with a mixing gas;
a plurality of mixing gas feed lines arranged to feed said mixing gas to said mixing region;
wherein each of said mixing gas feed lines includes a valve so that the amount of mixing gas admitted into the mixing region by each of the feed lines can be independently controlled;
wherein each of said mixing gas feed lines are branched off from a main header and preferably the reagent feed line is also branched off from said main header;
said mixing region includes a mixing device, said mixing device including distributors arranged in parallel, each of said distributors being fed individually and separately by one of the mixing gas feed lines;
the system further comprises a programmable controller or a distributed control system configured to adjust the aperture or closure of each of said valves as a function of the load of the converter, so that the flow rate of mixing gas fed to each of said distributors is individually controlled.

Particularly preferably, said distributors are ring distributors or toroidal distributors arranged concentrically.

### FIGURES

FIG. 1 shows an embodiment of the reactor system.

FIG. 2 shows another embodiment of the reactor system.

FIG. 3 shows a further embodiment of the reactor system.

FIG. 4 shows an embodiment of the flow distributors of the mixing device.

### DETAILED DESCRIPTION OF THE PREFERRED EMBODIMENTS

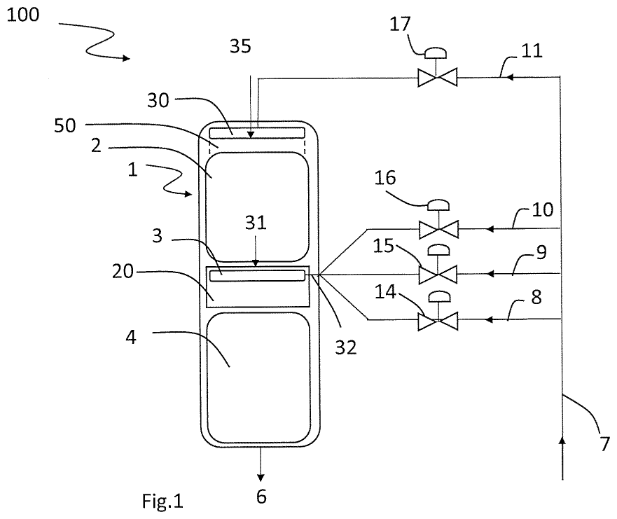

FIG. 1 is a schematic representation of a reactor system 100 of an ammonia synthesis process. The reactor system 100 comprises an ammonia converter 1, a reagent feed line 35 connected to said converter 1 and a plurality of mixing gas feed lines 8, 9, 10, 11.

The reagent feed line 35 and the mixing gas feed lines 8, 9, 10, 11 carry an ammonia make-up gas containing hydrogen and nitrogen (fresh reagent).

The converter 1 is a multibed converter including for example a first catalytic bed 2 and a second catalytic bed 4. In practical case, the converter 1 may include a greater number of beds, for example three or four.

The mixing gas feed lines 8, 9, 10, 11 are branched off from a main header 7. The line 35 may also be branched off from the same header 7. The mixing gas line 11 is in fluid communication with the first catalytic bed 2 via a mixing section 50 of the converter. In this embodiment, for simplicity of representation, the mixing section 50 is shown outside the catalytic bed 2. However, in some configurations, the mixing region 50 can be arranged over the catalyst and contained together with the catalytic bed 2 inside a catalytic basket.

The flow rate in the first mixing gas line 11 can be regulated by a valve 17 whilst the flow rate in the remaining mixing gas lines 8, 9 and 10 can be regulated by respective valves 14, 15, 16.

The mixing gas lines 8, 9, 10 are arranged to feed a portion of the mixing gas from the main header 7 into an inter-bed mixing region 20 downstream of the first catalytic bed 2 and upstream the second catalytic bed 4.

The valves 14, 15, 16 and 17 are operated by a programmable controller or by a distributed control system (not represented in figure) which through its implemented logic regulate the breakdown of the makeup gas carried by line 7 between the mixing gas lines 8, 9, 10, 11.

The inter-bed mixing region 20 is configured to receive a partially converted effluent 31 leaving the first catalytic bed and one or more stream of mixing gas carried by the mixing lines 8, 9 and 10. The mixing gas carried by the mixing lines 8, 9, 10 is fed to the inter-bed coolant section 20 via a flow distributor 3 after traversing a manifold 32.

A plurality of flow sensor devices (e.g. flow meter devices) and pressure sensor devices (e.g. pressure transducers and differential pressure cells) are distributed across the mixing gas feed lines to generate the input signals to the programmable interface control unit or to the distributed control system. Pressure should be preferably detected at least on the mixing line 11, upstream and downstream the valve 17, and flow rate should be measured at least on the main header 7.

The programmable interface control unit or the distributed control system elaborates the input signals received from the sensor devices and provides an output signal to the actuators of the valves 14, 15, 16, 17 to regulate the opening of said valves by adjusting the plug's displacement.

Each valve is provided with its own actuator therefore the flow rate passing through each valve can be adjusted independently accordingly to the output signal provided by the programmable control unit or by the distributed control system.

When the reactor system 100 operates at full capacity, the valves 14, 15, 16, 17 located on the mixing gas line 8, 9, 10, 11 are assumed to be in a mostly opened position in order to assure a proper flow control. When the flow rate carried by the line 7 drops, the flow rate and flow velocity circulating in mixing gas lines 14 to 17 are reduced as well. However, such reduction in not necessarily linear because the resistance to flow (pressure drop) is proportional to the square of the velocity in conventional size pipes.

To restore the flow velocity of the gas entering the mixing section 20 of the converter 1 to the target velocity range two situations are envisaged depending on the flow rate entering in the main header 7.

Specifically, if the reduction in flow rate circulating in line 7 is substantial, at least one of the valves 14, 15, 16 located on the lines 8, 9, 10 closes so that no mixing gas is circulating in the lines where the valves are shut.

Additionally, the valve 17 located on the first mixing line 11 partially closes to create an additional pressure drop on said first line thus compensating the not linear dependence between flow velocity and pressure so that the flow velocity of each portion of mixing gas entering the mixing section 20 is maintained in a target velocity range.

Alternatively, when the reduction in flow rate circulating in the header 7 is only marginal, the valves 14, 15, 16 located on the mixing lines 8, 9, 10 partially close simultaneously so that the mixing gas is distributed in equal portions between said mixing lines 8, 9, 10.

Likewise, the valve 17 located on the first mixing line 11 partially closes to create an additional pressure drop on the first line, and thus maintaining the flow velocity of the gas entering the mixing section 20 to a constant velocity range ideal for establishing good mixing conditions.

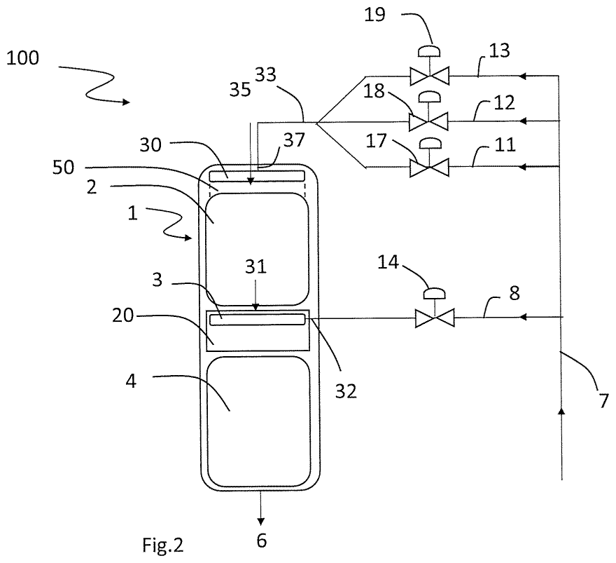

FIG. 2 illustrates another embodiment of the present invention wherein the flow velocity of the mixing gas entering the mixing section 50 arranged above the first catalytic bed 2 is maintained in a target flow velocity range. In this embodiment a good mixing can be established between the reagent gas entering the reagent feed line 35 and the mixing gas carried by the mixing lines 11, 12, 13.

During a partial load event, the valves 17, 18, 19 located on the mixing lines 11, 12, 13 partially close simultaneously or at least one of them close completely depending on the amount of mixing gas entering the main header 7. Instead, the valve 14 located on the mixing line 8 closes only partially, to create an additional pressure drop on the line 8 and compensate for the nonlinear relationship between pressure drop and flow velocity.

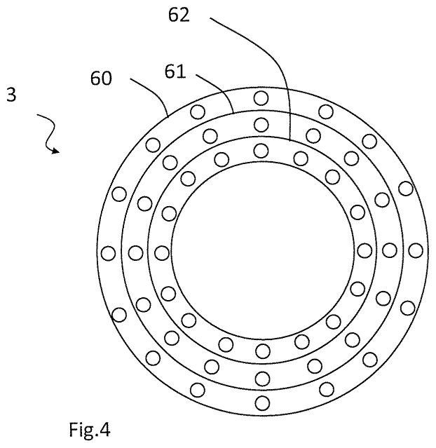

The mixing gas carried by the mixing lines 11, 12, 13 can be distributed in the mixing section 50 of the converter via the mixing device 30 comprising three flow distributors (FIG. 4). The three flow distributors are in fluid communication with the respective mixing lines 11, 12, 13 via a manhole 37 comprising three concentrically arranged tubes, so that each flow distributor is fed separately by one of the lines 11, 12 or 13.

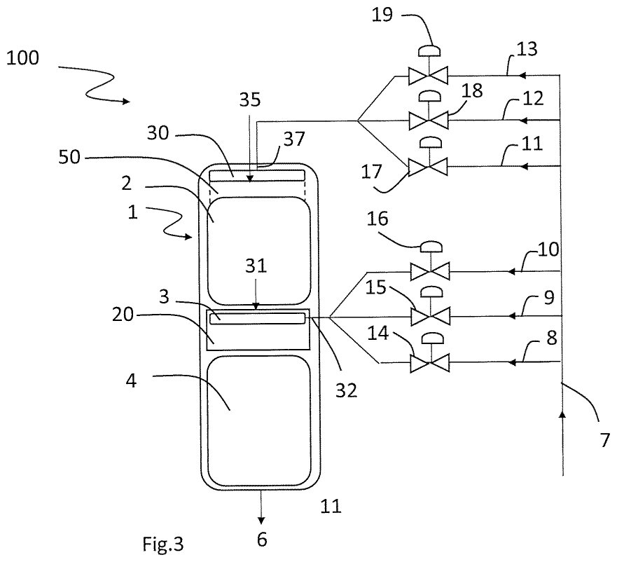

FIG. 3 shows another embodiment of the present invention, wherein the mixing gas feed lines comprise a plurality of lines 8 to 13 communicating with the two mixing sections 20 and 50. Lines 8 to 10 are connected to the mixing section 20 and lines 11 to 13 are connected to the mixing section 50.

In this embodiment, the flow regulator devices 14 to 19 allow maintaining the flow velocity of the mixing gas entering the two mixing sections 20, 50 in a target flow velocity range independently on the flow rate of the mixing gas entering the main header 7.

FIG. 4 shows an embodiment wherein the mixing device 3 comprises toroidal flow distributors 60, 61, 62. Each of said flow distributors is fed individually and separately by one of the feed lines 8, 9 and 10. It is noted that line 32 of FIG. 3 denotes an assembly of three coaxial pipes so that each stream of line 8, line 9 and line 10 can be separately fed to the distributor 60, 61 and 62 respectively. For example line 8 is connected to the distributor 60 via a first pipe of the coaxial assembly 32; line 9 is connected to the distributor 61 via a second pipe of the assembly and line 10 is connected to the distributor 62 via a third pipe of the assembly.

Said flow distributors 60, 61 and 62 are concentrically arranged and provided with circular openings to discharge into the mixing region each portion of the mixing gas circulating in the mixing lines 8, 9, 10.

1-14. (canceled)

15. A reactor system, comprising:
a catalytic converter;
a reagent feed line connected to the catalytic converter;
wherein the catalytic converter includes a multibed converter having a plurality of catalytic beds, wherein the catalytic converter further includes a plurality of mixing regions, wherein a number of the plurality of mixing regions is equal to a number of the plurality of catalytic beds, each of the plurality of mixing regions being arranged upstream of a respective catalytic bed to mix the inlet feed of the catalytic bed with a mixing gas; and
one or more mixing gas feed lines arranged to feed the mixing gas to the plurality of mixing regions, each of the one or more mixing regions being connected to a respective at least one of the one or more respective mixing gas feed lines;
wherein each of the one or more mixing gas feed lines includes at least one flow regulator device so that an amount of mixing gas admitted into the mixing region by each of the one or more mixing gas feed lines is independently controllable.

16. The reactor system according to claim 15, wherein the one or more mixing gas feed lines are branched off from a main header and also the reagent feed line is branched off from the main header.

17. The reactor system according to claim 15, wherein each of the plurality of mixing regions includes a mixing device connected to respective one of the one or more mixing gas feed lines.

18. The reactor system according to claim 17, wherein the mixing device includes a plurality of flow distributors, each of the plurality of flow distributors being connected in fluid communication with a respective mixing gas feed line.

19. The reactor system according to claim 18, wherein the plurality of flow distributors are concentrically arranged and preferably have the shape of rings or toroids.

20. The reactor system according to claim 18, wherein the mixing device is connected to the one or more mixing gas feed lines via a plurality of pipes arranged coaxially, wherein each of the plurality of pipes individually connects one gas feed line with a respective distributor of the mixing device.

21. The reactor system according to claim 15, wherein at least one flow regulator device includes a valve.

22. The reactor system according to claim 15, further comprising a programmable controller or a distributed control system configured to adjust the flow rate through the at least one flow regulator device as a function of a load of the catalytic converter.

23. The reactor system according to claim 22, wherein the control system is configured to adjust the opening degree of the valves.

24. The reactor system according to claim 15, wherein the one or more mixing gas feed lines are quench lines configured to carry a mixing gas having a temperature lower than a temperature of the feed stream of the catalytic bed, so that the mixing gas can be used as a quench gas.

25. The reactor system according to claim 15, wherein the catalytic converter is adapted for synthesis of ammonia or the catalytic converter is adapted for synthesis of methanol.

26. The reactor system according to claim 15, further comprising a heat exchanger arranged downstream a catalytic bed to remove heat from the effluent of the catalytic bed.

27. A method to adjust the flow rate circulating in the reactor system according to claim 15, wherein the flow rate of the mixing gas fed into the mixing regions of the converter is adjusted by the flow regulator device(s) as a function of the load of the catalytic converter.
